(19) United States
(12) Patent Application Publication
(10) Pub. No.: US 2024/0060153 A1
(43) Pub. Date: Feb. 22, 2024

(54) Acidomyces Acidothermus and Its Application in Leaching Copper-containing Pollutants from Waste Circuit Boards

(71) Applicant: Jiangsu University of Technology, Changzhou (CN)

(72) Inventors: Xiafei YIN, Changzhou (CN); Xuan SHAN, Changzhou (CN); Guobin LIANG, Changzhou (CN); Wei LIN, Changzhou (CN); Yanchen HUA, Changzhou (CN); Long YE, Changzhou (CN); Zilu ZHANG, Changzhou (CN); Xian ZHANG, Changzhou (CN); Feifei WANG, Changzhou (CN); Yuancheng LIU, Changzhou (CN); Wei SHAO, Changzhou (CN); Shilong SHAO, Changzhou (CN); Quanfa ZHOU, Changzhou (CN); Pengju WANG, Changzhou (CN)

(21) Appl. No.: 18/495,183

(22) Filed: Oct. 26, 2023

Related U.S. Application Data
(63) Continuation of application No. PCT/CN2022/140280, filed on Dec. 20, 2022.

(51) Int. Cl. C22B 7/00; C22B 15/00; C12N 1/20

(52) U.S. Cl. CPC C22B 7/006; C22B 15/00; C12N 1/20

(57) ABSTRACT

The present disclosure discloses a strain of Acidomyces acidothermus and its application in leaching copper-containing pollutants from waste circuit boards, belonging to the technical field of bio-leaching. According to the present disclosure, a strain of A. acidothermus is separated from the sludge of a Changzhou sewage treatment plant; and the strain can normally grow under an acidic condition, and copper contained in a system can be leached out in a form of copper ions by adding the strain into waste or pollutants containing heavy metal copper, so that the metal copper in the waste or pollutants is effectively treated. The treatment process is simple, the requirements for the environment and the technology are low, and the treated waste liquid does not contain high-concentration chemical reagents, which facilitates subsequent waste liquid treatment; and therefore, the A. acidothermus is suitable for industrial application.

### REFERENCE TO SEQUENCE LISTING

The instant application contains a Sequence Listing in XML format as a file named“YGHY-2023-28 SEQ.xml”, created on Oct. 7, 2023, of 5 kB in size, and which is hereby incorporated by reference in its entirety.

### TECHNICAL FIELD

The present disclosure relates to a strain of Acidomyces acidothermus and its application in leaching copper-containing pollutants from waste circuit boards, belonging to the technical field of bio-leaching.

### BACKGROUND

Copper is an essential trace element for all living organisms, as it is a main component of the respiratory enzyme complex cytochrome c oxidase. Copper is a component of blood pigment hemocyanin in mollusks and crustaceans and is replaced by iron-complexed hemoglobin in fishes and other vertebrates. In humans, copper is mainly present in the liver, muscles, and bones. Adult bodies contain 1.4 to 2.1 milligrams of copper per kilogram of body weight. When a large amount of heavy metal copper remains in the human body, it is very easy to cause a burden on the organs in the body, especially the liver and gallbladder. When these two organs have problems, the metabolism maintained in the body will be disordered.

Waste circuit boards contain a large amount of heavy metals, and if discarded without treatment, they will have a serious impact on the environment. At present, heavy metal copper is mainly leached by water leaching, ammonia leaching, and acid leaching methods. The acid leaching method is mainly divided into inorganic acid leaching and organic acid leaching. The ammonia leaching method can solve the problem of high acid consumption in the acid leaching method, and the production cost of the ammonia leaching method is relatively low.

At present, the microbial leaching of copper is related to the leaching of heavy metal copper from ores by Thiobacillus ferrooxidans, but the current research on bioleaching heavy metal copper in ores is still in the exploratory stage, and it is also necessary to further study the leaching efficiency and cultivate efficient strains that can adapt to industrial production.

### SUMMARY

According to the present disclosure, a strain of A. acidothermus is separated from the sludge of a Changzhou sewage treatment plant, and the strain can be used for leaching metal copper, thus providing an effective biological treatment method for treating the metal copper.

The first object of the present disclosure is to provide a strain of A. acidothermus, and the A. acidothermus has been preserved in China Center for Type Culture Collection (CCTCC) on May 25, 2021, with a preservation number CCTCC No.22431.The preservation address is Institute of Microbiology, Chinese Academy of Sciences, Beijing, China.

The second object of the present disclosure is to provide a product containing the A. acidothermus.

In one embodiment, the product includes, but is not limited to, a microbial preparation, a sewage treatment agent, a biocatalyst, or an oxidant.

In one embodiment, the product is prepared from the A. acidothermus or from the fermentation broth of the A. acidothermus.

In one embodiment, the product also contains one or more strains of Pseudomonas, Bacillus, Streptococcus, Serratia, Thiobacillus, Actinomyces, and Aspergillus.

The third object of the present disclosure is to provide a method for leaching copper, the method includes: adding the A. acidothermus or the product to a copper-containing system, copper-containing pollutants, and/or copper-containing waste, so as to leach the copper out from the pollutant or waste.

In one embodiment, the copper-containing pollutants include waste circuit boards, waste copper etching solutions, copper-containing sludge, and/or copper-containing organic pollutants.

In one embodiment, the A. acidothermus is cultured into bacterial liquid with an OD₆₀₀ of 0.1-1.5, and then reacted according to an amount of adding 10-100 mL of bacterial liquid per gram of pollutants, or reacted according to an amount of adding 25-250 mL of bacterial liquid per gram of copper elemental substance, to leach the copper out from a waste circuit board.

In one embodiment, the A. acidothermus is cultured at a pH of 2.5-3.5 under the conditions of 140-180 rpm, and 30-35° C. until the OD₆₀₀ is 0.4-1.5; and preferably, the OD₆₀₀ is 0.8.

In one embodiment, a reaction is carried out under the conditions of 25-35° C. and 0-250 rpm; and preferably, the reaction is carried out under the environment of 140-180 rpm and 30-35° C.

In one embodiment, the reaction time is not less than 1 h; and preferably, the reaction time is 1-8 h.

The fourth object of the present disclosure is to provide application of the A. acidothermus or the product containing the A. acidothermus in the treatment of copper-containing pollutants or copper-containing waste.

In one embodiment, the copper-containing pollutants or copper-containing waste includes waste circuit boards, copper-containing wastewater, copper-containing powder, waste copper etching solutions, copper-containing sludge, and/or copper-containing organic pollutants.

Beneficial effects of the present disclosure: According to the present disclosure, the A. acidothermus is separated from the sludge of the Changzhou sewage treatment plant; and the strain can normally grow under an acidic condition, and copper contained in a system can be leached out in a form of copper ions by adding the strain into waste or pollutants containing heavy metal copper, so that the metal copper in the waste or pollutants is effectively treated. The treatment process is simple, the requirements for the environment and the technology are low, and the treated waste liquid does not contain high-concentration chemical reagents, which facilitates subsequent waste liquid treatment.

### Biological Material Preservation

The strain provided by the present disclosure, with the taxonomic name Acidomyces acidothermus, has been preserved in the China General Microbiological Culture Collection Center (CGMCC) on May 25, 2021, with a preservation number CGMCC No.22431, and the preservation address is Institute of Microbiology, Chinese Academy of Sciences, No. 3, Courtyard No. 1, Beichen West Road, Chaoyang District, Beijing.

### BRIEF DESCRIPTION OF FIGURES

FIG. 1 shows diagrams presenting the colony morphology and cell morphology of A. acidothermus; and

FIG. 2 is an electrophoretogram of A. acidothermus, where A is a gel diagram of a product amplified by using an ITS primer pair, and B is a gel diagram of a product amplified by using an NS primer pair.

### DETAILED DESCRIPTION

Mediums involved in the following examples are as follows:

LB liquid medium: 5 g·L⁻¹ yeast powder, 10 g·L⁻¹ tryptone, 10 g·L⁻¹ sodium chloride, and 1000 mL of distilled water.

LB solid medium: 5 g·L⁻¹ yeast powder, 10 g·L⁻¹ tryptone, 10 g·L⁻¹ sodium chloride, 1000 mL of distilled water, and 20 g of agar.

The acidic LB solid medium or the acidic LB medium described in the following examples is prepared by adjusting the pH of the LB solid medium to 3.

The calculation of a leaching rate involved in the following examples:

Calculation of a copper leaching rate:

Leaching rate=(C*A*V)/m.

C denotes concentration of copper ions; A denotes dilution ratio; V denotes volume of bacterial liquid; and m denotes initial mass of copper in copper-containing wastewater from waste circuit boards.

The initial mass of copper in the wastewater from the circuit board is determined by spectrophotometry, which is 40% of the mass of powder from the waste circuit board (1 g of the powder from the waste circuit board contains 0.4 g of copper).

Technical terms:

Sludge: The term “sludge” is taken from a Changzhou sewage treatment plant, with a density of 0.027 g·mL⁻¹ and a pH of 7.66.

Copper-containing system: Copper exists in an elemental form in the system.

Copper-containing pollutants: including high-concentration organic pollutants produced during the process of printed circuit boards, which contain high-concentration copper.

Copper-containing waste: including waste copper etching solutions and copper-containing sludge produced in the production process of circuit boards.

Powder from waste circuit boards: The term “powder from waste circuit boards” derives from the Changzhou sewage treatment plant. It is a powdered copper-containing substance obtained by crushing the waste circuit boards through a crusher. The mass of copper is 40% of the total mass of the powder.

The present disclosure will be further described below with reference to the examples, but the embodiments of the present disclosure are not limited thereto.

### EXAMPLE 1: STRAIN SCREENING

(1) 10 mL of sludge with a density of 0.027 g·mL⁻¹ obtained from the Changzhou sewage treatment plant was taken and placed into 90 mL of the LB liquid medium, and cultured for 4-6 d in an environment of 140-180 rpm and 30-35° C.;

(2) the bacterial liquid obtained in step (1) was taken and incubated into 100 mL of a new LB liquid medium in an inoculation amount being 5-10% by volume ratio, and cultured for 4-6 d in an environment of 140-180 rpm and 30-35° C.;

(3) the bacterial liquid obtained in step (2) was taken and incubated into 100 mL of a new LB liquid medium in an inoculation amount being 5-10% by volume ratio, and cultured for 4-6 d in an environment of 140-180 rpm and 30-35° C.;

(4) 5μL of the bacterial liquid obtained in step (3) was taken and added to a sterilized LB liquid medium, and cultured for 4-6 d in a shaker under the conditions of 140-180 rpm and 30-40° C.; 100 μL of the obtained liquid was pipetted with a pipette tip, and an acidic LB solid medium was coated with the pipetted liquid;

(5) the acidic LB solid medium coated with the bacterial liquid was placed in an incubator and incubated at 37° C. for 1-2 d, and the morphology of colonies was observed; a small number of strains were selected from each of the colonies and inoculated into the acidic LB liquid mediums; after 4-6 d of cultivation, the acidic LB solid mediums were coated with the obtained bacterial liquid;

(6) step (5) was repeated for a plurality of times until a single strain was obtained from each of the acidic LB solid mediums; and

(7) a single strain was selected from the medium in which the single strain was grown, inoculated into 100 mL of a new acidic LB liquid medium, and cultured in an environment of 140-180 rpm and 30-35° C. for 4-6 d; and the OD₆₀₀ value of the bacterial liquid was measured, and the desired bacterial liquid was obtained when the OD₆₀₀ value reached 0.8.

### EXAMPLE 2: IDENTIFICATION OF STRAINS

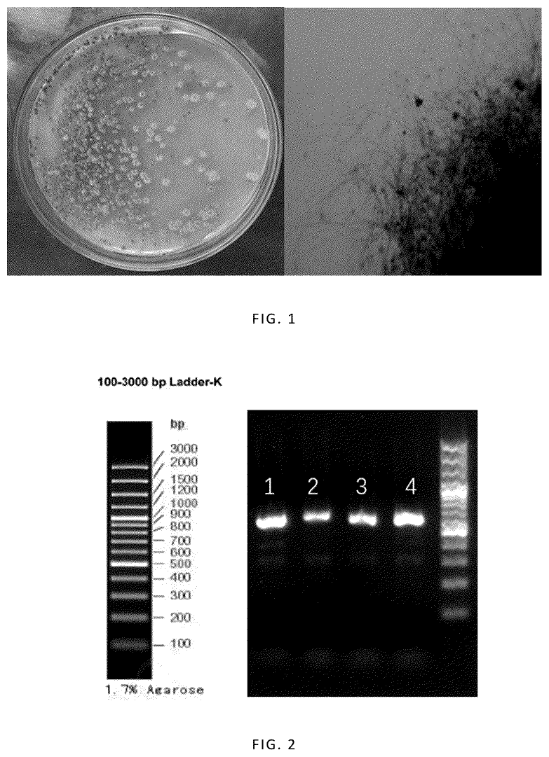

(1) The bacterial liquid obtained in Example 1 was taken, and the morphological characteristics of a strain were observed by using an optical microscope. The results are as shown in FIG. 1. The bacterial cells of the strain are velvety, and the colonies of the strain are round and opaque.

(2) The strain was sent to Shanghai Sangon Bioengineering Co., Ltd. for identification by 16SrDNA method.

After amplification using ITS and NS universal primers, the primers and amplification system were as follows: the amplified bands were detected by electrophoresis. The electropherogram is as shown in a lane 3 in FIG. 2 (with a band size of about 950 bp). The bands were sequenced, and the sequencing results were compared on a BLAST. After comparison, the amplified sequence of the strain was 100% similar to that of Acidomyces, so that the strain was identified as Acidomyces acidothermus, and sent to the strain preservation Center for preservation.

\textcircled{1} Primers used:

| (SEQ ID NO: 1) | |
|---|---|
| ITS1: | TCCGTAGGTGAACCTGCGG, |
| (SEQ ID NO: 2) | |
| ITS4: | TCCTCCGCTTATTGATATGC; |
| (SEQ ID NO: 3) | |
| NS1: | GTAGTCATATGCTTGTCTC, |
| (SEQ ID NO: 4) | |
| NS6: | GCATCACAGACCTGTTATTGCCTC. |

\textcircled{2} PCR amplification reaction system:

| 10 × PCR Buffer | |
|---|---|
| dNTP (each 10 mM) | 12.5 μl in total |
| Taq Plus DNA Polymerase (5 U/μl) | |
| 50 mM MgSO₄ | |
| Primer F (10 μM) | 1 μl |
| Primer R (10 μM) | 1 μl |

\textcircled{3} PCR reaction conditions:

| | | | |
|---|---|---|---|
| 95° C. | 5 | min | |
| 94° C. | 30 | s | 30 cycles |
| 57° C. | 30 | s | |
| 72° C. | 90 | s | |
| 72° C. | 10 | min | |

### EXAMPLE 3: APPLICATION OF A. acidothermus IN COPPER LEACHING

Influence of adding different amounts of bacteria liquid on copper leaching effect

1. Influence of 20 mL of bacterial liquid on leaching effect

(1) The screened strain was inoculated into an acidic LB liquid medium, and cultured under the conditions of 140-180 rpm and 30-35° C. until the OD₆₀₀ value of the bacterial liquid is 0.8;

(2) 20 mL of the bacterial liquid obtained in step (1) was taken, 1 g of powder from waste circuit boards was added to the bacterial liquid, the mixture was stirred on a magnetic stirrer at a speed of 180 r/min, a sample was pipetted every 4 h, and the copper ion concentration in the sample was measured;

(3) the sample liquid was centrifuged, and the supernatant obtained by a high-speed low-temperature centrifuge was filtered to obtain the required clear and transparent leachate;

(4) after the leachate obtained in step (3) was diluted 250 times, the concentration of copper ions in the bacterial liquid was measured by ICP when adding 20 mL of the bacterial liquid; and

(5) the leaching rate of copper was calculated. The results are as shown in Table 1. The strain has a good leaching effect on the copper ions in the waste circuit boards. After 4 h of treatment, the leaching rate of copper can reach 1.3161%.

TABLE 1
The concentrations of copper ions at different times after
adding 20 ml of A. acidothermus bacterial liquid

| | Time/h | | | |
|---|---|---|---|---|
| | 0 | 1 | 4 | 8 |
| Concentration of | 0 | 0.136 | 263.2126 | 124.7404 |
| copper ions/mg · L⁻¹ | | | | |
| Leaching rate of copper | 0% | 0.0174% | 1.3161% | 0.6237% |

2. Influence of 30 mL of bacterial liquid on leaching effect

According to the above steps, 30 mL of bacterial liquid was taken, 1 g of powder from waste circuit boards was added to the bacterial liquid, the mixture was stirred on a magnetic stirrer at a speed of 180 r/min, and a sample was pipetted every 4 h; the sample liquid was centrifuged, and the supernatant obtained by a high-speed low-temperature centrifuge was filtered to obtain the required clear and transparent leachate; and after the obtained leachate was diluted 250 times, the concentration of copper ions in copper-containing wastewater from the waste circuit boards was measured by ICP.

The results are as shown in Table 2. After the amount of the bacterial liquid is increased, the leaching effect of copper ions is significantly improved, and the leaching rate of copper in waste circuit boards reaches 3.1693% or above.

TABLE 2
The concentrations of copper ions at different times after
adding 30 mL of A. acidothermus bacterial liquid

| | Time/h | | | |
|---|---|---|---|---|
| | 0 | 1 | 4 | 8 |
| Concentration of | 0 | 1344.911 | 2325.756 | 1056.427 |
| copper ions/mg · L⁻¹ | | | | |
| Leaching rate of copper | 0% | 4.0347% | 6.9773% | 3.1693% |

3. Influence of 40 mL of bacterial liquid on leaching effect

According to the above steps, 40 mL of bacterial liquid was taken, 1 g of powder from waste circuit boards was added to the bacterial liquid, the mixture was stirred on a magnetic stirrer at a speed of 180 r/min, and a sample was pipetted every 4 h; the sample liquid was centrifuged, and the supernatant obtained by a high-speed low-temperature centrifuge was filtered to obtain the required clear and transparent leachate; and after the obtained leachate was diluted 250 times, the concentration of copper ions in copper-containing wastewater from the waste circuit boards was measured by ICP.

The results are as shown in Table 3. After 40 mL of the bacterial liquid is added to 1 g of the powder from the waste circuit boards and cultured for a period of time, the leaching rate of copper ions in the powder from the waste circuit boards is further improved, reaching up to 10.6877%.

TABLE 3
The concentrations of copper ions at different times after
adding 40 ml of A. acidothermus bacterial liquid

| | Time/h | | | |
|---|---|---|---|---|
| | 0 | 1 | 4 | 8 |
| Concentration of | 0 | 2210.362 | 2671.936 | 1546.85 |
| copper ions/mg · L⁻¹ | | | | |
| Leaching rate of copper | 0% | 8.8414% | 10.6877% | 6.1874% |

4. Influence of 50 mL of bacterial liquid on leaching effect

According to the above steps, 50 mL of bacterial liquid was taken, 1 g of powder from waste circuit boards was added to the bacterial liquid, the mixture was stirred on a magnetic stirrer at a speed of 180 r/min, and a sample was pipetted every 4 h; the sample liquid was centrifuged, and the supernatant obtained by a high-speed low-temperature centrifuge was filtered to obtain the required clear and transparent leachate; and after the obtained leachate was diluted 250 times, the concentration of copper ions in copper-containing wastewater from the waste circuit boards was measured by ICP.

The results are as shown in Table 4. By adding 1 g of the powder from the waste circuit boards to 50 mL of the bacterial liquid and reacting for 1-8 h, the leaching rate of copper ions can reach 5.7149%.

TABLE 4
The concentrations of copper ions at different times after
adding 50 mL of A. acidothermus bacterial liquid

| | Time/h | | | |
|---|---|---|---|---|
| | 0 | 1 | 4 | 8 |
| Concentration of | 0 | 1142.973 | 749.0226 | 730.5562 |
| copper ions/mg · L⁻¹ | | | | |
| Leaching rate of copper | 0% | 5.7149% | 3.9701% | 3.6528% |

5. Influence of 100 mL of bacterial liquid on leaching effect

According to the above steps, 100 mL of bacterial liquid was taken, 1 g of powder from waste circuit boards was added to the bacterial liquid, the mixture was stirred on a magnetic stirrer at a speed of 180 r/min, and a sample was pipetted every 4 h; the sample liquid was centrifuged, and the supernatant obtained by a high-speed low-temperature centrifuge was filtered to obtain the required clear and transparent leachate; and after the obtained leachate was diluted 250 times, the concentration of copper ions in copper-containing wastewater from the waste circuit boards was measured by ICP.

The results are as shown in Table 5. By adding 1 g of the powder from the waste circuit boards to 100 mL of the bacterial liquid, the leaching rate of copper ions can reach 18.535%.

TABLE 5
The concentrations of copper ions at different times after
adding 100 ml of A. acidothermus bacterial liquid

| | Time/h | | | |
|---|---|---|---|---|
| | 0 | 1 | 4 | 8 |
| Concentration of | 0 | 1027.579 | 1835.333 | 190.9762 |
| copper ions/mg · L⁻¹ | | | | |
| Leaching rate of copper | 0% | 10.2758 | 18.353% | 1.9098% |

### EXAMPLE 4: A METHOD FOR LEACHING COPPER

A strain was inoculated into an acidic LB medium, and cultured under the conditions of 140-180 rpm and 30-35° C. until the ( )am value of the bacterial liquid was 0.8; 10-100 mL of the bacterial liquid with the OD₆₀₀ of 0.8 was taken, and 1 g of powder from waste circuit boards was added to the bacterial liquid, the mixture was stirred on a magnetic stirrer at a speed of 180 r/min for 0-8 h; and after the reaction, the concentration of copper ions could range from 0.136 mg/L to 2671.936 mg/L, and the leaching rate of copper ions could reach 18%.

### EXAMPLE 5: PREPARATIONn OF A PRODUCT CONTAINING A. acidothermus

200-600 μL of A. acidothermus was inoculated in 10-30 mL of an acidic LB liquid medium and activated for 2-3 generations at 30° C. When the number of viable bacteria in A. acidothermus reached 10⁸ cfu/mL or above, centrifugation was carried out at 8000 rpm for 15 min. After the supernatant was removed, bacterial cells were taken and freeze-dried, which could be mixed with other bacterial powder to prepare a mixed bacterial agent. The other bacterial powder includes Pseudomonas, Bacillus, Streptococcus, Serratia, Thiobacillus, Actinomyces, and Aspergillus.

### EXAMPLE 6: LEACHING COPPER BY ACID LEACHING

Specific operation steps:

(1) 20-50 mL of sulfuric acid was separately taken and added to 1 g of copper-containing wastewater of powder from waste circuit boards, and the mixture was stirred on a magnetic stirrer at a speed of 180 r/min.

(2) The liquid obtained in step (1) was pipetted for centrifugation, and the supernatant obtained by a high-speed low-temperature centrifuge was filtered to obtain the required clear and transparent leachate.

(3) The concentration of copper ions in 1 g of the copper-containing wastewater of the powder from the waste circuit boards was measured by ICP using the leachate obtained in step (2) when different amounts of sulfuric acid were added.

(4) In step (3), the concentration of copper ions was measured from 1 to 8 h respectively.

(5) The leaching rate of copper was calculated:

Leaching rate=(C*A*V)/m, where

C denotes concentration of copper ions; A denotes dilution ratio; V denotes volume of sulfuric acid; and m denotes initial mass of copper in 1 g of powder from waste circuit boards.

TABLE 6

| The concentrations of copper ions at different | | |
|---|---|---|
| times after adding 20 ml of sulfuric acid | | |
| | Time/h | |
| | 2 | 3 |
| Concentration of copper | 0.70667 | 0.429725 |
| ions/mg · L⁻¹ | | |
| Leaching rate of copper | 0.8833% | 0.5372% |

TABLE 7

| The concentrations of copper ions at different | | |
|---|---|---|
| times after adding 50 ml of sulfuric acid | | |
| | Time/h | |
| | 4 | 8 |
| Concentration of | 557.4695 | 1042.1186 |
| copper ions/mg · L⁻¹ | 2.7873% | 5.2106% |
| Leaching rate of copper | | |

Although the present disclosure has been disclosed as above in exemplary examples, it is not intended to limit the present disclosure. Anyone familiar with this technology can make various changes and modifications without departing from the spirit and scope of the present disclosure. Therefore, the protection scope of the present disclosure shall be as defined in the Claims.

What is claimed is:

1. A microbial preparation, wherein the microbial preparation contains a strain of Acidomyces acidothermus, and the A. acidothermus has been preserved in China General Microbiological Culture Collection Center (CGMCC) on May 25, 2021, with a preservation number CGMCC No.22431.

2. A method for leaching copper, comprises adding the A. acidothermus or the microbial preparation according to claim 1 to a copper-containing system to leach the copper out from the system; and the copper-containing system is a waste circuit board.

3. The method according to claim 2, wherein the A. acidothermus is cultured into bacterial liquid with an OD₆₀₀ of 0.1-1.5, and then reacted according to an amount of adding 10-100 mL of bacterial liquid per gram of pollutants, or reacted according to an amount of adding 25-250 mL of bacterial liquid per gram of copper elemental substance, to leach the copper out from the waste circuit board.

4. The method according to claim 2, wherein the reaction is carried out under the conditions of 25-35° C. and 0-250 rpm.

5. Application of the A. acidothermus or the microbial preparation according to claim 1 in the treatment of copper-containing waste, wherein the application comprises: adding the A. acidothermus or the microbial preparation to a copper-containing system to leach the copper out from the system; the copper-containing system is a waste circuit board.


SEQUENCE LISTING (4 sequences)

SEQ ID NO: 1 - DNA, 19 bp, synthetic construct
  source 1..19: mol_type = other DNA
tccgtaggtgaacctgcgg

SEQ ID NO: 2 - DNA, 20 bp, synthetic construct
  source 1..20: mol_type = other DNA
tcctccgcttattgatatgc

SEQ ID NO: 3 - DNA, 19 bp, synthetic construct
  source 1..19: mol_type = other DNA
gtagtcatatgcttgtctc

SEQ ID NO: 4 - DNA, 24 bp, synthetic construct
  source 1..24: mol_type = other DNA
gcatcacagacctgttattgcctc